(19) United States
(12) Patent Application Publication
(10) Pub. No.: US 2019/0151274 A1
(43) Pub. Date: May 23, 2019

(54) COMPOUND FOR USE IN THE TREATMENT OF A BLOOD VESSEL DISEASE

(71) Applicant: Universitaet Potsdam, Potsdam (DE)

(72) Inventors: Salim Seyfried, Berlin (DE); Cécile Otten, Berlin (DE); Kristina Haehn, Berlin (DE)

(21) Appl. No.: 16/193,025

(22) Filed: Nov. 16, 2018

(30) Foreign Application Priority Data
Nov. 17, 2017 (EP) 17202432.5

(51) Int. Cl. A61K 31/203; A61K 31/11; A61K 31/167; A61K 31/07; A61K 45/06; A61P 9/00

(52) U.S. Cl. CPC A61K 31/203; A61K 31/11; A61P 9/00; A61K 31/07; A61K 45/06; A61K 31/167

(57) ABSTRACT

Object of the present invention are compounds for use in the treatment of a blood vessel disease, as well as pharmaceutical compositions comprising said compound, for use in the treatment of a blood vessel disease, and methods for treatment of blood vessel diseases. Described herein are compounds according to general formula I

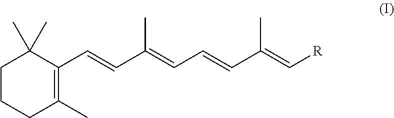

wherein R is selected from the group consisting of —CHO, —COOH, —CH₂OH and

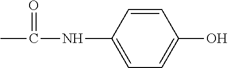

as well as the stereoisomers, enantiomers, diastereomers, the physiological acceptable salts thereof, as well as mixtures of said compounds, for use in the treatment of a blood vessel disease.

The present invention relates to a compound for use in the treatment of a blood vessel disease as well as a pharmaceutical composition comprising said compound and a method for treatment of a blood vessel disease.

Cerebral cavernous malformations (CCMs) are blood vessel diseases in humans that give rise to vascular lesions in the central nervous system, what in turn can cause epileptic seizures as well as hemorrhagic strokes.

Cavernous tumors occur sporadically or familial with autosomal dominant heredity. More than 90% of the inheritable cavernous tumors are based on variations in the CCM1, CCM2, or CCM3 gene. The gene product of CCM1 is the protein KRIT1 (Krev interaction trapped 1), and this is why the CCM1 gene is frequently also called KRIT1 gene. PDCD10 (Programmed cell death protein 10) is the gene product of CCM3, and this is why the CCM3 gene is frequently also called PDCD10 gene.

So far, the only method of treatment is the surgical intervention for removal or laser sclerotherapy of CCMs. But this treatment is associated with high strain on the persons affected. Furthermore, in many cases the large number of lesions (hundreds up to thousands lesions in one patient), the age of the patient (CCM3 diseases already occur in infants), as well as the affected brain area (for example brainstem or deep-seated lesions), is not treatable by the surgeon. In these cases it is a “fateful illness” for which a pharmacological intervention is urgently desired.

Object of the present invention is therefore to overcome the disadvantages of the state of the art and to provide a compound that can be used to pharmacologically treat blood vessel diseases.

The object is solved by providing a compound for use in the treatment of a blood vessel disease according to the features of the main claim. Preferred embodiments of the compound according to the invention are defined in the dependent subclaims.

An object of the present invention is a compound according to general formula I

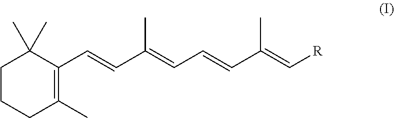

wherein R is selected from the group consisting of —CHO, —COOH, —CH₂OH and

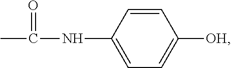

as well as the stereoisomers, enantiomers or diastereomers thereof, the physiological acceptable salts thereof, as well as mixtures of said compounds, for use in the treatment of a blood vessel disease.

Preferred is a compound according to the invention for use in the treatment of a blood vessel disease, wherein the blood vessel disease is selected from cerebral cavernous malformations, hemorrhagic strokes, hereditary hemorrhagic telangiectasias or arteriovenous malformations.

In the sense of the present invention, the blood vessel disease is not restricted to the listed diseases, but can also include similar diseases which have related molecular or physiological causes.

Especially preferred is a compound according to the invention for use in the treatment of a blood vessel disease, wherein the compound is present in an orally administrable form, wherein the orally administrable form is a capsule, a tablet, a suspension, a solution or a dragée, or that the compound is present in an intravenously administrable form, wherein the intravenously administrable form is a suspension or a solution, or that the compound is present in a subcutaneously administrable form, wherein the subcutaneously administrable form is a suspension, a solution or a pellet.

Furthermore a compound according to the invention for use in the treatment of a blood vessel disease is preferred, wherein the administration of the compound is carried out according to a dosing regime, wherein the dosing of the compound is defined depending on the age, sex and weight of a patient that shows a blood vessel disease, as well as on the type and severity of the blood vessel disease, and wherein the duration of the treatment is at least one day.

A further object of the present invention is a pharmaceutical composition for use in the treatment of a blood vessel disease, comprising a compound according to the invention or the physiological acceptable salts, stereoisomers, enantiomers and diastereomers thereof or mixtures thereof, and pharmaceutical acceptable excipients and/or carriers in combination with one or more substances, selected from the group consisting of indirubin-3-monoxime, indirubine, vitamin D, vitamin D3, rapamycin, statins, sulindac and its metabolites, RhoA, Rho kinase inhibitors, vascular growth factor inhibitors, PTC-209, Unc1999, Mek/Erk inhibitors, propranolol, propranolol hydrochloride and antioxidants.

In the sense of the present invention the statins can be selected from simvastatin and atorvastatin.

Rho kinase inhibitors in the sense of the invention can be fasudil as well as BA-1049 and BA2017.

In the sense of the present invention vascular growth factor (VEGF) inhibitors can be semaxanib (SU5416) as well as bevacizumab.

In the sense of the present invention XMD 8-92 is a Mek/Erk inhibitor.

Antioxidants in the sense of the present invention can be chosen from the group consistent of N-acetylcysteine, tempol, tranilast and avenantrhramide.

Especially preferred is a pharmaceutical composition according to the invention for use in the treatment of a blood vessel disease, wherein the blood vessel disease is selected from cerebral cavernous malformations, hemorrhagic strokes, hereditary hemorrhagic telangiectasias or arteriovenous malformations.

In particular preferred is a pharmaceutical composition for use in the treatment of a blood vessel disease, wherein the pharmaceutical composition is present in an orally administrable form, wherein the orally administrable form is a capsule, a tablet, a suspension, a dragée, or that the pharmaceutical composition is present in an intravenously administrable form, wherein the intravenously administrable form is a suspension or a solution, or that the pharmaceutical composition is present in a subcutaneously administrable form, wherein the subcutaneously administrable form is a suspension, a solution or a pellet.

Additionally preferred is a pharmaceutical composition for use in the treatment of a blood vessel disease, wherein the administration of the pharmaceutical composition is carried out according to a dosing regime, wherein the dosing of the compound is defined depending on the age, sex and weight of a patient that shows a blood vessel disease, as well as on the type and severity of the blood vessel disease, and the duration of the treatment is at least one day.

A further object of the present invention is a method for treatment of a blood vessel disease, wherein one administers to a patient, that shows at least one vascular lesion or is at the risk to develop a vascular lesion, a therapeutically effective amount of a compound according to the invention, or the physiologically acceptable salts, stereoisomers, enantiomers, diastereomers or mixtures thereof.

In the sense of the present invention the patient is a human being.

The therapeutically effective amount of the compound can, in the sense of the present invention, depend on the stage (acute or chronic disease) as well as possibly on the severity of the disease (measured on the basis of the number of CCM lesions and the occurrence of bleedings). In particular the occurrence of bleedings is considered in the field to be the acute status. Furthermore a dosage can have a prophylactic character.

For example a patient with only one CCM lesion needs less or a lower dosage of the compound according to the invention as a patient with two or more CCM lesions.

Preferred is a method according to the invention wherein the blood vessel disease is selected from cerebral cavernous malformations, hemorrhagic strokes, hereditary hemorrhagic telangiectasias or arteriovenous malformations.

Especially preferred is a method according to the invention wherein one determines before administration as well as after administration of the therapeutically effective amount of the compound the number of vascular lesions and/or the size of at least one vascular lesion, as well as the stage of the disease.

The stage of the disease defines in the sense of the present invention whether it is an acute or chronic disease. Furthermore a dosage can have a prophylactic character.

Additionally preferred is a method according to the invention, wherein the detection of the number and/or the size of the at least one vascular lesion is performed by means of magnetic resonance tomography.

Preferred is a method according to the invention wherein the compound is administered orally in form of a capsule, a tablet, a suspension, a solution or a dragée, or intravenously in form of a suspension or solution, or subcutaneously in form of a suspension or a solution or a pellet.

Especially preferred is a method according to the invention, wherein the method comprises a step wherein a patient is identified that shows at least one vascular lesion or is at the risk of developing at least one vascular lesion.

Furthermore preferred is a method according to the invention, wherein for identifying a patient that shows a cerebral cavernous malformation or the risk to develop a cerebral cavernous malformation, a mutation in at least one gene of the patient is identified, which is associated with the cerebral cavern-cavernous malformation, wherein the gene is selected from CCM2, KRIT1 or PDCD10.

Especially preferred is also a method, wherein for identifying a patient that shows a hereditary hemorrhagic telangiectasia or the risk to develop a hereditary hemorrhagic telangiectasia, a mutation in at least one gene of the patient is identified, which is associated with the hereditary hemorrhagic telangiectasia, wherein the gen is selected from ALK1, ENG, SMAD4, GDF2 or ACVRL1.

The present invention will be illustrated in more detail with the attached Figures.

It shows:

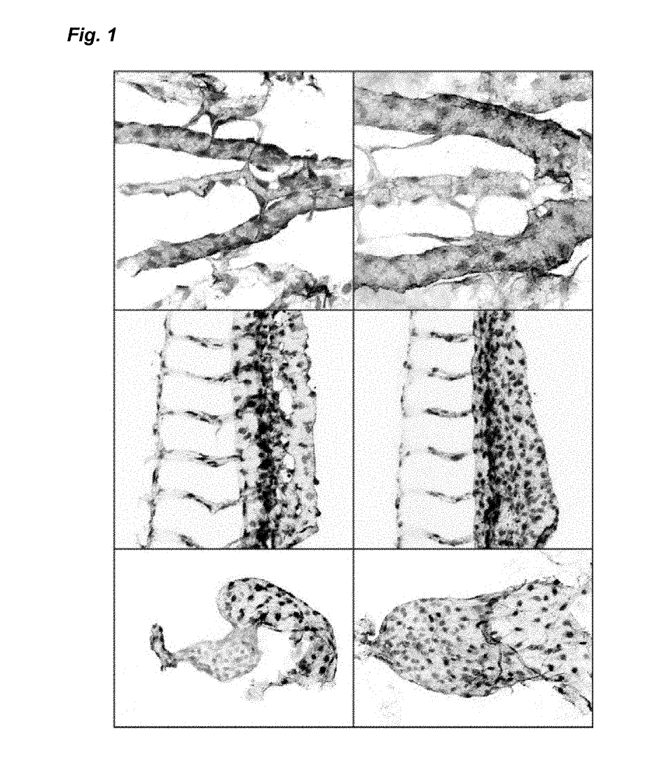

FIG. 1 photos that give an overview over the CCM mutant phenotypes in the zebrafish embryo compared to the wild-type;

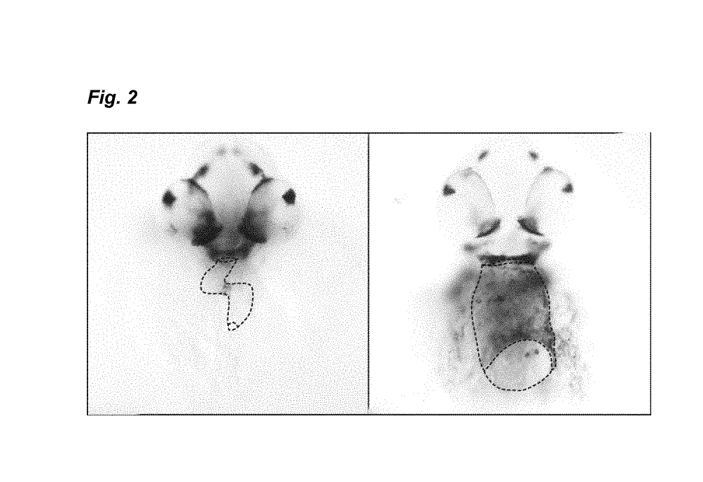

FIG. 2 microscopic images that show the effect of the CCM2 mutation on the expression of aldehyde dehydrogenase 1 in the zebrafish;

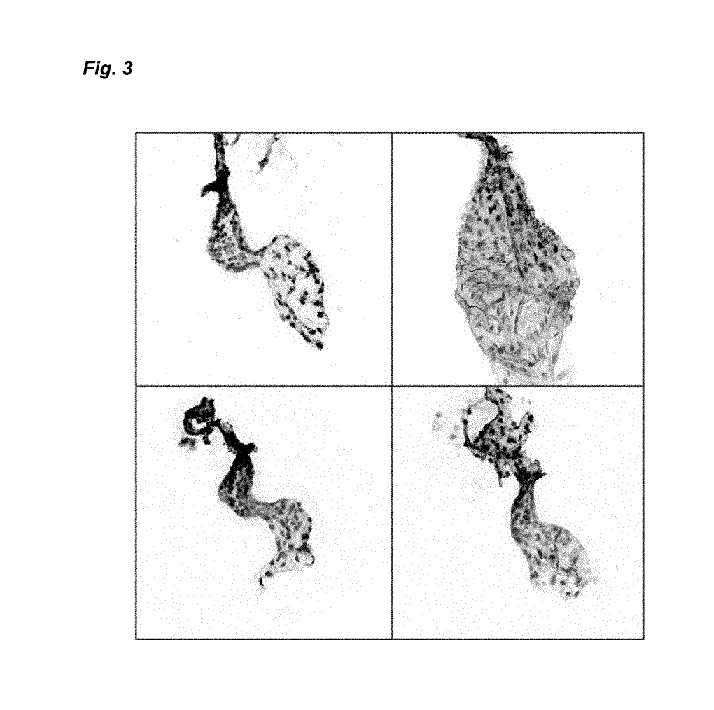

FIG. 3 microscopic images of the heart of a wild-type zebrafish embryo and of a CCM2 mutant after the treatment with retinoic acid;

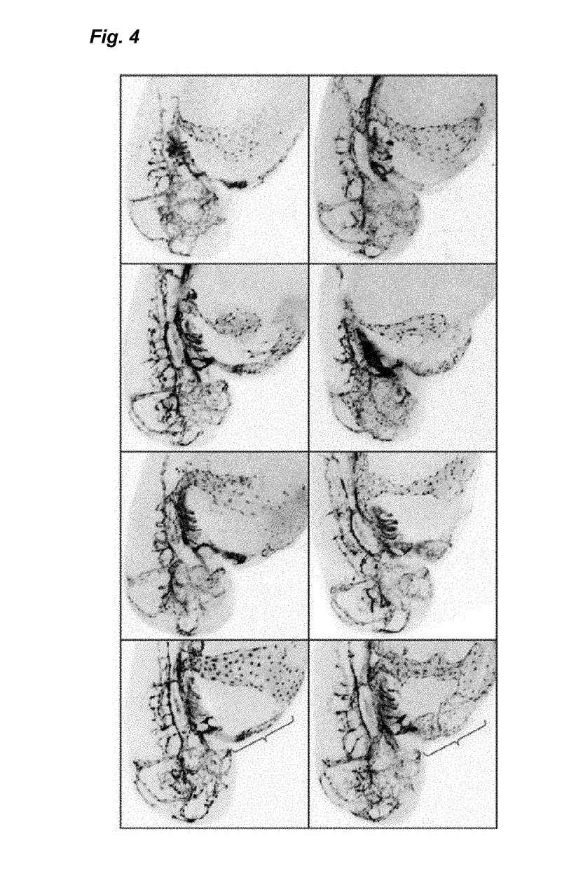

FIG. 4 microscopic side images of the head and heart of a wild-type zebrafish embryo and of a CCM2 mutant after a treatment with retinal, retinol or talarozole; and

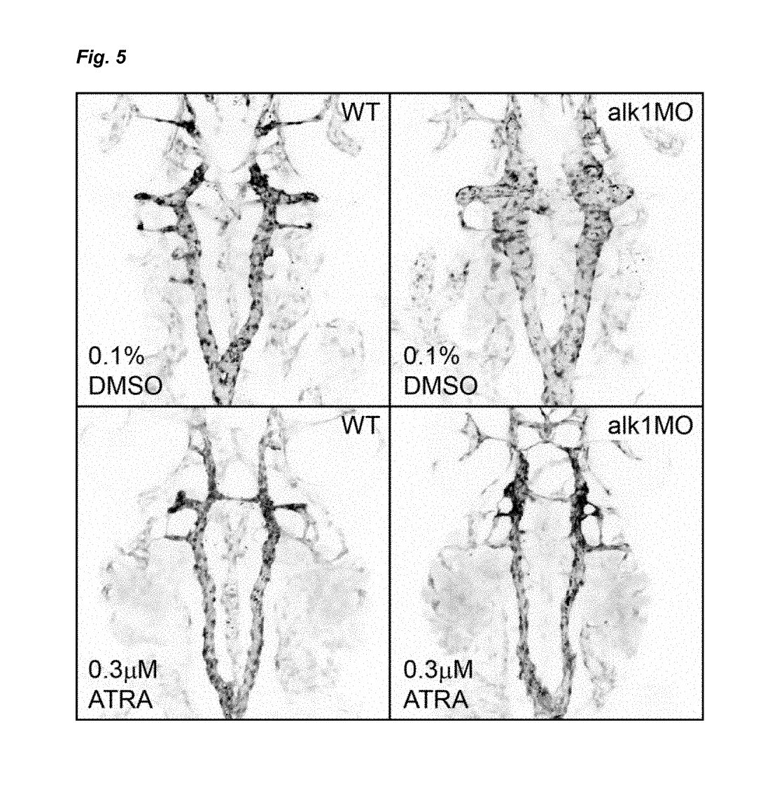

FIG. 5. microscopic images of the aorta of a wild-type zebrafish and of an ALK1 mutant after the treatment with all-trans-retinoic acid.

A compound of general formula I is retinoic acid as well as the precursors or derivatives thereof.

Compared to the compounds of the state of the art, these compounds have the advantage that they can specifically and significantly treat blood vessel diseases in humans and animals. Furthermore it is advantageous that these compounds show a pharmacologic suppressing effect.

Thus it is also advantageous that these compounds can be in particular used for the treatment of cerebral cavernous malformations. Cerebral cavernous malformations are especially characterized by the formation of vascular lesions in the central nervous system. Therefore the pharmacologically suppressing effect by the application of the compounds according to the invention is especially advantageous for the treatment of cerebral cavernous malformations.

The pharmaceutical composition of the present invention is prepared with the conventionally used pharmaceutical technical excipients and additives according to the desired kind of application in the suitable dosage.

Capsules that contain the pharmaceutical composition according to the invention can be prepared for example by mixing the intended ingredients with inert carriers, such as cellulose, microcrystalline cellulose, hydroxypropyl methyl cellulose, lactose or sorbitol and encapsulating in gelatin capsules.

Corresponding tablets can, for example, be obtained by mixing of the active ingredients with known excipients, for example inert diluents, such as dextrose, sugar, sorbitol, mannitol, polyvinyl pyrrolidone, disintegrators, such as corn starch or alginic acid, binders, such as starch or gelatin, lubricants, like magnesium stearate or talc, and/or means for achieving a depot effect, such as carboxylpolymethylene, carboxylmethylcellulose, cellulose acetate phtalate or polyvinyl acetate. The tablets can also consist of several layers.

In a corresponding manner, dragées can be prepared by coating cores, prepared in analogy to the tablets, with excipients usually used in dragée coats, such as, for example, polyvinyl pyrrolidone or shellac, gum arabic, talc, titanium dioxide, or sugar. Here, the dragée shell can also consist of several layers, wherein the excipients mentioned above for the tablets can be used.

Solutions or suspensions with the active ingredients of the pharmaceutical composition according to the invention can contain, in addition, substances that improve taste, such as saccharin, cyclamate, or sugar, as well as, for example, flavoring substances, such as vanilla or orange extract. In addition, they can contain suspending agents, such as sodium carboxymethylcellulose, or preservatives, such as p-hydroxybenzoates.

Water, oils or other suitable fluids can be used as solvents or suspending agents for solutions or suspensions.

Furthermore the solutions can also be parenteral formulations like solutions for injection.

The preparation of the pharmaceutical composition follows the general principles for preparation of pharmaceutical products. The preparation of the pharmaceutical composition to the invention is in itself known and is described in handbooks known to the person skilled in the art, such as, for example Hager's Handbuch [Hager's Handbook], (5th) 2, 622-1045; List et al., Arzneiformenlehre [Drug Forms], Stuttgart: Wiss. Verlagsges. 1985; Sucker et al., Pharmazeutische Technologie [Pharmaceutical Technology], Stuttgart: Thieme 1991; Ullmann's Enzyklopädie [Ullmann's Encyclopedia] (5th) A 19 241-271; Voigt, Pharmazeutische Technologie [Pharmaceutical Technology], Berlin: Ullstein Mosby 1995.

Surprisingly the inventors found that the use of a compound of formula I suppresses the development of the CCM mutant phenotype in the zebrafish embryo. This approach will also allow for a pharmacological intervention in humans, what will be in particular advantageous when no surgical intervention is possible. For example, when lesions are located deep in the brainstem or the lesions already occur in infants. Additionally advantageous is that a reduction in the rate of growth and/or in the number of CCM lesions in a patient also reduces the occurrence of symptoms that are associated with CCM, like for example epilepsy, hemorrhage, and focal neurological deficits.

Furthermore the use of a compound according to formula I is also advantageous in the use for treatment of other blood vessel diseases, which are based on similar molecular mechanisms as the CCM disease. These dis-diseases include, inter alia, hemorrhagic strokes, hereditary hemorrhagic telangiectasias or arteriovenous malformations. But this list is only an exemplary list of blood vessel diseases and is no restriction of the present invention.

Additionally advantageous with the use of a compound according to formula I for the treatment of a blood vessel disease is that no long-term therapy is necessary, but already with short-term treatment periods significant improvements of the disease are induced.

The following compounds are therefore, according to the invention, especially preferred embodiments:

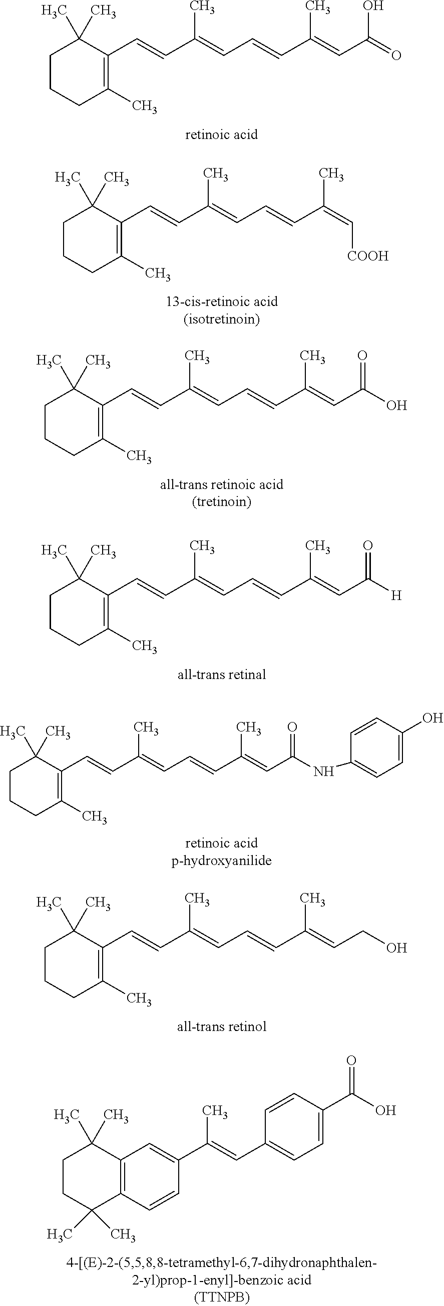

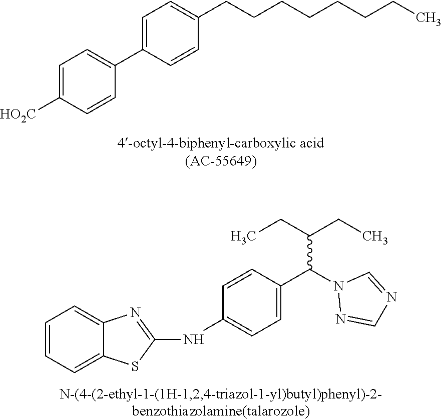 [SMILES: CCCCCCCCc1ccc(-c2ccc(C(=O)O)cc2)cc1 ; CCC(CC)C(c1ccc(Nc2nc3ccccc3s2)cc1)n1cncn1]

The following examples illustrate the invention in more detail without restricting the scope of the invention.

### EXAMPLE 1

The images displayed in FIG. 1 show an overview comparing a wild-type zebrafish embryo and a zebrafish embryo with a CCM2 mutant phenotype. The three images in the left column show the wild-type zebrafish embryo, whereas the images in the right column show the CCM2 mutant phenotype at the age of two days after insemination. Additionally the images at the bottom of the figure show the heart, the images in the middle show the caudal plexus and intersegmental vessels, and the upper images show the aorta.

The images of the zebrafish embryo of the CCM2 mutant phenotype show cardiovascular deficits in the respective regions, compared to the respective region of the wild-type zebrafish embryo.

### EXAMPLE 2

In FIG. 2 microscopic images are shown, which show the expression of retinaldehyde dehydrogenase 2 (RALDH2) in the zebrafish embryo.

The left image shows the expression of RALDH2 in the heart of a wild-type zebrafish, and the right image shows the expression in the heart of a zebrafish with CCM2 mutant phenotype.

RALDH2, also referred to as family member A2, is a family member of aldehyde dehydrogenase 1 and in humans is encoded by gene ALDH1A2. RALDH2 is an enzyme which catalyzes the conversion of retinaldehyde to retinoic acid.

The images in FIG. 2 thus show that the expression of the RALDH2 enzyme in the heart of a CCM2 mutant zebrafish, compared to the expression in the heart of a wild-type zebrafish, is highly increased. It is known that the deficiency of retinoic acid leads to the activation of the RALDH2 expression.

This indicates that the CCM2 mutants suffer from retinoic acid deficiency.

### EXAMPLE 3

In FIG. 3 microscopic images are shown, which illustrate the results of a treatment with retinoic acid in a wild-type zebrafish and in a CCM mutant.

Within a period of time of 16-51 hours after insemination of zebrafish eggs, one group of zebrafishes was treated with a 0.1 μM solution of retinoic acid, and one group of zebrafishes was treated with a 0.1% solution of dimethyl sulfoxide (DMSO). The 0.1% DMSO solution is used as control.

In FIG. 3 the left images exemplary show the hearts of treated wild-type zebrafishes, and the right images show the hearts of treated CCM2 mutants. The upper row shows the results of the treatment with 0.1% DMSO solution, and the lowest row shows the results of the treatment with a 0.1 μM solution of retinoic acid.

From the histological illustrations in FIG. 3 it can be derived that the heart of the CCM2 mutant, which was treated with DMSO as control, is balloon-like enlarged, compared to the heart of the wild-type zebrafish. Furthermore FIG. 3 shows unambiguously that the treatment of the zebrafish with CCM2 mutant phenotype with retinoic acid ensures that the balloon-like enlargement of the heart is strongly alleviated, and thus the CCM2 mutant phenotype as a whole is alleviated and is very much alike the phenotype of the wild-type.

Thus the examples show the strong pharmacological suppressing effect of a compound of formula I, especially of retinoic acid, on the development of the cardiovascular phenotypes in CCM mutants in the zebrafish model.

### EXAMPLE 4

In FIG. 4, exemplary, the effects of active ingredients on the development of treated CCM2 mutant hearts, in comparison with the heart of the wild-type zebrafish and a CCM2 mutant after treatment with a 0.1% DMSO solution, are shown (the region of the heart is marked by black brackets).

Within a period of time of 16-51 hours after insemination of zebrafish eggs, the treatment with different active ingredients took place. The treatment was performed with 0.1% DMSO (lowest row), 1 μM retinal (second lowest row), 1 μM retinol (second row counted from top) and 10 μM talarozole (top row). The left column shows the results after the treatment in the hearts of wild-type zebrafishes with the different active ingredients, and the right column shows the results in the hearts of CCM2 mutants after the treatment with the different active ingredients.

It can be derived from the respective sub-images that the heart of the CCM2 mutant that was treated with DMSO as control is balloon-like enlarged, compared with the heart of the wild-type zebrafish. Furthermore FIG. 4 shows unambiguously that the treatment of the CCM2 mutant phenotype with the listed active ingredients ensures that the balloon-like enlargement of the heart is very much alleviated and thus as a whole the CCM2 mutant phenotype is alleviated and is very much alike the phenotype of the wild-type.

These examples prove the strong pharmacological suppression of the CCM2 mutant heart phenotype in the zebrafish model.

### EXAMPLE 5

FIG. 5 shows the effects of all-trans retinoic acid (ATRA) treatment on the development of the ALK1 mutants' aorta, in comparison with the normal aorta of a wild-type zebrafish and an ALK1 mutant aorta after treatment with a 0.1% DMSO control solution.

The zebrafish ALK1 mutant is in a gene that is defective in some patients that suffer from HHT (hereditary hemorrhagic telangiectasia). The ALK1 mutant phenotype is an enlarged lateral dorsal aorta (LDA).

The treatment with the different active ingredients took place, between 24-51 hours after insemination of zebrafish eggs. The treatment was performed with 0.1% DMSO (first row) and 0.3 μM ATRA (second row). The left column shows the results after the treatment in the hearts of wild-type zebrafishes, which were treated with DMSO or ATRA while the right column shows the effects of DMSO or ARTRA treatment on the lateral dorsal aorta of ALK1 mutants.

It can be derived from the images that the overgrowth of the aorta is suppressed in the ALK1 mutant after treatment with ATRA.

1. A compound of formula I 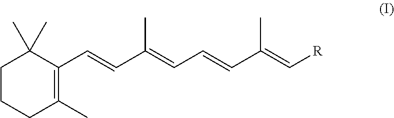
wherein R is selected from the group consisting of —CHO, —COOH, CH₂OH and 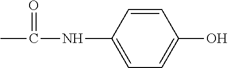
as well as the stereoisomers, enantiomers or diastereomers thereof, the physiological acceptable salts thereof, as well as mixtures of said compounds, for use in the treatment of a blood vessel diseases.

2. The compound for use in the treatment of a blood vessel disease, according to claim 1, characterized in that the blood vessel disease is selected from cerebral cavernous malformations, hemorrhagic strokes, hereditary hemorrhagic telangiectasias or arteriovenous malformations.

3. The compound for use in the treatment of a blood vessel disease, according to claim 1, characterized in that the compound is present in an orally administrable form, wherein the orally administrable form is a capsule, a tablet, a suspension, a solution or a dragée, or that the compound is present in an intravenously administrable form, wherein the intravenously administrable form is a suspension or a solution, or that the compound is present in a subcutaneously administrable form, wherein the subcutaneously administrable form is a suspension, a solution or a pellet.

4. The compound for use in the treatment of a blood vessel disease, according to claim 1, characterized in that the administration of the compound is carried out according to a dosing regime, wherein the dosing of the compound is defined depending on the age, sex and weight of a patient that shows a blood vessel disease, as well as on the type and severity of the disease, and wherein the duration of the treatment is at least one day.

5. A pharmaceutical composition for use in the treatment of a blood vessel disease, comprising a compound according to claim 1, or the physiological acceptable salts thereof, stereoisomers, enantiomers, diastereomers, or mixtures thereof, and pharmaceutical acceptable excipients and/or carriers in combination with one or more substances, selected from the group consisting of indirubin-3-monoxime, indirubine, vitamin D, vitamin D3, rapamycin, statins, sulindac and its metabolites, RhoA, Rho kinase inhibitors, vascular growth factor inhibitors, PTC-209, Unc1999, Mek/Erk inhibitors, propranolol, propranolol hydrochloride and antioxidants.

6. The pharmaceutical composition for use in the treatment of a blood vessel disease, according to claim 5, characterized in that the blood vessel disease is selected from cerebral cavernous malformations, hemorrhagic strokes, hereditary hemorrhagic telangiectasias or arteriovenous malformations.

7. The pharmaceutical composition for use in the treatment of a blood vessel disease, according to claim 5, characterized in that the pharmaceutical composition is present in an orally administrable form, wherein the orally administrable form is a capsule, a tablet, a suspension, a solution or a sugar-coated tablet, or that the pharmaceutical composition is present in an intravenously administrable form, wherein the intravenously administrable form is a suspension or a solution, or that the pharmaceutical composition is present in a subcutaneously administrable form, wherein the subcutaneously administrable form is a suspension, a solution or a pellet.

8. The pharmaceutical composition for use in the treatment of a blood vessel disease, according to claim 5, characterized in that the administration of the pharmaceutical composition is carried out according to a dosing regime, wherein the dosing is defined depending on the age, sex and weight of a patient that shows a blood vessel disease, as well as on the type and severity of the blood vessel disease, and wherein the duration of the treatment is at least one day.

9. A method for treatment of a blood vessel disease, wherein one administers to a patient, that shows at least one vascular lesion or is at the risk to develop a vascular lesion, a therapeutically effective amount of a compound according to claim 1, or the physiologically acceptable salts, stereoisomers, enantiomers, diastereomers or the mixtures thereof.

10. The method, according to claim 9, characterized in that the blood vessel disease is selected from cerebral cavernous malformations, hemorrhagic strokes, hereditary hemorrhagic telangiectasias or arteriovenous malformations.

11. The method, according to claim 9, characterized in one determines, before administration and after administration of the therapeutically effective amount of the compound, the number of vascular lesions and/or the size of at least one vascular lesion, as well as the stage of the disease.

12. The method, according to claim 11, characterized in that detection of the number and/or the size of the at least one vascular lesions is performed by means of magnetic resonance tomography.

13. The method, according to claim 9, characterized in that the compound is administered orally in form of a capsule, a tablet, a suspension, a solution or a dragée, or intravenously in form of a suspension or solution, or subcutaneously in form of a suspension or a solution or a pellet.

14. The method, according to claim 9, characterized in that the method comprises a step wherein a patient is identified that shows at least one vascular lesion or is at the risk of developing at least one vascular lesion.

15. The method, according to claim 14, characterized in that for identifying a patient that shows a cerebral cavernous malformation or the risk to develop a cerebral cavernous malformation, a mutation in at least one gene of the patient is identified, which is associated with the cerebral cavernous malformation, wherein the gene is selected from CCM2, KRIT1 or PDCD10.

16. The method, according to claim 14, characterized in that for identifying a patient that shows a hereditary hemorrhagic telangiectasia or the risk to develop a hereditary hemorrhagic telangiectasia, a mutation in at least one gene of the patient is identified, which is associated with the hereditary hemorrhagic telangiectasia, wherein the gen is selected from ALK1, ENG, SMAD4, GDF2 or ACVRL1.
